(19) United States
(12) Patent Application Publication
(10) Pub. No.: US 2018/0162911 A1
(43) Pub. Date: Jun. 14, 2018

(54) POLYPEPTIDES FOR THE DIAGNOSIS AND THE TREATMENT OF C3 NEF ASSOCIATED C3 GLOMERULOPATHY

(71) Applicants: INSERM (INSTITUT NATIONAL DE LA SANTÉ ET DE LA RECHERCHE MÉDICALE), Paris (FR); UNIVERSITÉ PARIS DESCARTES, Paris (FR); UNIVERSITÉ PIERRE ET MARIE CURIE (PARIS 6), Paris (FR); UNIVERSITÉ PARIS DIDEROT - PARIS 7, Paris (FR); ASSISTANCE PUBLIQUE-HÔPITAUX DE PARIS (APHP), Paris (FR)

(72) Inventors: Véronique FREMEAUX-BACCHI, Paris (FR); Lubka ROUMENINA, Paris (FR); Maria-Chiara MARINOZZI, Paris (FR)

(21) Appl. No.: 15/737,358

(22) PCT Filed: Jun. 17, 2016
(86) PCT No.: PCT/EP2016/064087

§ 371 Date: Dec. 18, 2017

(30) Foreign Application Priority Data
Jun. 19, 2015 (EP) 15305952.2

(51) Int. Cl. C07K 14/00; C07K 7/08; A61P 13/12; G01N 33/564; G01N 33/68

(52) U.S. Cl. CPC C07K 14/00; C07K 7/08; A61P 13/12; A61K 38/00; G01N 33/6854; C07K 2319/00; G01N 33/564

(57) ABSTRACT

The present invention relates to polypeptides for the diagnosis and treatment of C3 NeF associated C3 Glomerulopathy. In particular, the present invention is defined by the claims. In particular, the present invention relates to a polypeptide that is capable of inhibiting the binding of C3 NeF to C3 convertase and which comprises a first segment which consists of n consecutive amino acids selected in a first amino acid sequence set forth in SEQ ID NO:1 fused to a second segment which consists of n′ consecutive amino acids selected in a second amino acid sequence set forth in SEQ ID NO:2, wherein n and n′ represent integer number, n and n′≥3 and n+n′≥10.

### FIELD OF THE INVENTION

The present invention relates to polypeptides for the diagnosis and treatment of C3 NeF associated C3 Glomerulopathy.

### BACKGROUND OF THE INVENTION

The C3 Glomerulopathy is a chronic renal disease with 50% of progression to end-stage renal disease after 10 years. C3 Glomerulopathy has been divided into dense deposit disease (DDD) and Glomerulonephritis with predominant C3 deposits (C3GN). This disease is correlated with the alternative complement pathway dysregulation and is associated in more of 70% of the cases with the presence of an autoantibody, the C3 Nephritic Factor (C3NeF). C3NeF was described for the first time in 1969 (Spitzer et al. Science 1969) and after it was characterized as an Immunoglobulin G capable to bind and stabilize the convertase of the alternative complement pathway (Daha et al. J Imm 1976), which in normal condition is a very unstable complex. It has also been suggested that C3NeF avoid the mechanisms of regulation of the C3 convertase (Weiler et al. PNAS 1976, Paixao-Cavalcante et al. KI 2012). As consequence, C3NeF leads to a massive cleavage of C3 and to the alternative pathway activation. C3NeFs are heterogeneous as some are associated with C3 consumption in the fluid phase and other are not; some can enhance the C5 consumption and the terminal pathway activation and others—no (Mollness et al. Clin Exp Imm 1986). Some experimental data in human and mice suggest that C3 Glomerulopathy is a properdin-dependent disease. It has been shown that some C3NeF require Properdin to stabilize the convertase and some do not (Tanuma et al. Clin Imm 1990). This heterogeneity suggests the presence of different C3NeFs with different functional activity and different consequences in-vivo, hypothesis that has not been widely studied so far. Recent studies showed that properdin influences the intraglomerular localization of C3 in a mice model of complete CFH deficiency. To define whether C3NeF binds to C3b, Bb or to both in the C3bBb complex, a study from Daha et al. in 1981 investigated the binding and the stabilization of homologous and heterologous cell-bound convertases prepared with human and rat C3 or FB. Nine out ten C3NeF were capable to bind only C3bhuBbhu and C3bratBbhu but not C3bhuBbrat and C3bratBbrat suggesting that these antibodies bind a specific human epitopes on the Bb portion exposed after its interaction with C3b (Daha and Van Es, 1981). Therefore the transfert of the disease is not possible.

C3 NeF was also associated with acquired lipodystrophy with or without GC3 Barraquer-Simons syndrome), a rare form of partial lipodystrophy characterized by gradual onset of bilaterally symmetrical subcutaneous fat loss from the face, neck, upper extremities, thorax, and abdomen but sparing the lower extremities. As there is no known single effective therapy for C3G, a variety of different therapeutic approaches are currently in use. Angiotensin-converting enzyme inhibitors (ACEI) or angiotensin II receptor blocker (ARB) are prescribed to most patients for their antiproteinuric and nephroprotective effect. The successful use of plasma therapy [plasma exchange (PE) or plasma infusions] is reported in some cases. Several authors have reported the use of plasmapheresis in DDD, but it was regarded as successful therapy only in some of these reports. Other potential strategies to reduce C3Nef (e.g., using rituximab) have only been reported in single cases or have shown only variable effects. Eculizumab (anti-C5 monoclonal antibody) has been given to a small number of patients, with variable success and the potential therapeutic effect of eculizumab in C3G patients is discussed controversial. This demonstrates a significant unmet need for the effective treatment of C3 NeF associated C3 Glomerulopathy.

### SUMMARY OF THE INVENTION

The present invention relates to polypeptides for the diagnosis and treatment of C3 NeF associated C3 Glomerulopathy. In particular, the present invention is defined by the claims.

### DETAILED DESCRIPTION OF THE INVENTION

The inventors confirm that C3NeF activity is heterogeneous since their results demonstrate that some C3NeFs activate C3 without affecting terminal pathway activation while other C3NeFs clearly are associated with C5-9 activation. The inventors found that IgG of 22 out of the 55 tested patients with histological evidence of C3G stabilized both the C3bBb and the C3bBbP convertases but 16 and 6 patients' IgG stabilized only the C3bBb or the C3bBbP convertases respectively. They identified two types of C3Nefs with different binding sites on the C3 convertase. The inventors use the term of C3NeF for all IgG capable to stabilize the C3 convertases C3bBb and C3bBbP. C3NeF of one of the groups was found to bind at the Properdin binding site and to compete with it, sharing the same effect (Properdin like C3NeF). The majority of C3NeF bind in an area, adjacent to the Properdin binding site and have an additive effect on the C3 convertase stabilization (Properdin independent C3 NeF). The inventors provide evidence that the cooperation between Properdin and C3NeF drives the C5 cleavage in C3 glomerulopathy as only C3NeFs that stabilize the C3bBb and the C3bBbP convertases increased significantly the levels of sC5b9 in patients plasma. Lastly using peptides and functional assays the inventors identified the binding site of C3NeF on the C3 convertase. The results shed new light on the pathogenesis of C3NeF related kidney disease and have direct therapeutic implications for human C3 glomerulopathy patients.

The inventors also show that one sub type of C3NeF and properdin, which both increase the AP activity, share the same binding site on the C3bBb convertase. By functional assays, they demonstrated that three peptides inhibits the binding of patients' IgG on the C3 convertase to induce their functional effect. In cases of Properdin-enhancer C3 NeF the peptides neutralizing Properdin binding will stop the amplification of the C3 convertase stabilization and limits the deleterious effect of the C5 activation. Lastly peptides neutralizing Properdin binding will inhibit the Properdin-dependant C3 NeF binding in this context.

Accordingly one object of the present invention relates to a polypeptide that is capable of inhibiting the binding of C3 NeF to C3 convertase and which comprises a first segment which consists of n consecutive amino acids selected in a first amino acid sequence set forth in SEQ ID NO:1 (GQDEENQKQCQDLGAFTESMVVF) fused to a second segment which consists of n′ consecutive amino acids selected in a second amino acid sequence set forth in SEQ ID NO:2 (LKHDEYNIENLQKTVWD), wherein n and n′ represent integer number, n and n′≥3 and n+n′≥10.

In some embodiments, the polypeptide of the present invention comprises at least 10, 11, 12, 13, 14, 15, 16, 17, 18; 19; 20; 21; 22; 23; 24; 25; 26; 27; 28; 29; 30; 31; 32; 33; 34; 35; 36; 37; 38; 39; or 40 amino acids. In some embodiments, the polypeptide of the present invention comprises less than 50 amino acids. In some embodiments, the polypeptide of the present invention comprises less than 30 amino acids. In some embodiments, the polypeptide of the present invention comprises less than 25 amino acids. In some embodiments, the polypeptide of the present invention comprises less than 20 amino acids. In some embodiments, the polypeptide of the present invention comprises less than 15 amino acids.

The functional properties of the polypeptide of the present invention, i.e. inhibition of the binding of C3 NeF to C3 convertase, could typically be assessed in any functional assay as described in EXAMPLE.

In some embodiments, the first segment consists of 3; 4; 5; 6; 7; 8; 9; 10; 11; 12; 13; 14; 15; 16; 17; 18; 19; 20; 21; 22; or 23 consecutive amino acids selected in the first amino acid sequence set forth in SEQ ID NO:1.

In some embodiments, the second segment consists of 3; 4; 5; 6; 7; 8; 9; 10; 11; 12; 13; 14; 15; 16; or 17 consecutive amino acids selected in the first amino acid sequence set forth in SEQ ID NO:2.

In some embodiments, the first and second segments are fused directly or via a spacer. As used herein, the term “directly” means that the (first or last) amino acid at the terminal end (N or C-terminal end) of the first segment is fused to the (first or last) amino acid at the terminal end (N or C-terminal end) of the second segment. In other words, in this embodiment, the last amino acid of the C-terminal end of said firs segment is directly linked by a covalent bond to the first amino acid of the N-terminal end of said second segment, or the first amino acid of the N-terminal end of said polypeptide is directly linked by a covalent bond to the last amino acid of the C-terminal end of said heterologous polypeptide. As used herein, the term “spacer” refers to a sequence of at least one amino acid that links the first segment to the second segment. Such a spacer may be useful to prevent steric hindrances.

In some embodiments, the polypeptide of the present invention consists of an amino acid sequence selected in Table A:

TABLE A
polypeptides that are suitable for inhibiting
the binding of C3 NeF to C3 convertase

| | | | SEQ |
|---|---|---|---|
| | | | ID |
| name | mer | sequence | NO: |
| short1 | 19 | GAFTESMVVFLKHDEYNIE | 3 |
| mix1 | 25 | KQCQDLGAFTESMVVFKATYPKIWV | 4 |
| pep1 | 34 | GQDEENQKQCQDLGAFTESMVVFNI | 5 |
| | | ENLQKTVWD | |
| Pep1C3bExt | 25 | GENQKQCQDLGAFTESMVVFLKHDE | 6 |
| Pep1BbExt | 22 | SMVVFLKHDEYNIENLQKTVWD | 7 |
| Essential | 13 | SMVVFNLQKTVWD | 10 |
| Essentiel2 | 17 | GAFTESMVVFNLQKTVWD | 11 |

The polypeptides of the present invention may be produced by any technique known per se in the art, such as, without limitation, any chemical, biological, genetic or enzymatic technique, either alone or in combination. For instance, knowing the amino acid sequence of the desired sequence, one skilled in the art can readily produce said polypeptides, by standard techniques for production of amino acid sequences. For instance, they can be synthesized using well-known solid phase method, typically using a commercially available peptide synthesis apparatus (such as that made by Applied Biosystems, Foster City, Calif.) and following the manufacturer's instructions. Alternatively, the polypeptides of the present invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques. Polypeptides of the present invention can be used in an isolated (e.g., purified) form or contained in a vector, such as a membrane or lipid vesicle (e.g. a liposome).

A further object of the present invention relates to a nucleic acid molecule encoding for the polypeptide of the present invention. As used herein, the term “nucleic acid molecule” has its general meaning in the art and refers to a DNA or RNA molecule. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fiuorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine. These nucleic acid sequences can be obtained by conventional methods well known to those skilled in the art. Typically, said nucleic acid is a DNA or RNA molecule, which may be included in a suitable vector, such as a plasmid, cosmid, episome, artificial chromosome, phage or viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Any expression vector for animal cell can be used. Examples of suitable vectors include pAGE107 (Miyaji et al., 1990), pAGE103 (Mizukami and Itoh, 1987), pHSG274 (Brady et al., 1984), pKCR (O'Hare et al., 1981), pSG1 beta d2-4 (Miyaji et al., 1990) and the like. Other examples of plasmids include replicating plasmids comprising an origin of replication, or integrative plasmids, such as for instance pUC, pcDNA, pBR, and the like. Other examples of viral vectors include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478. Examples of promoters and enhancers used in the expression vector for animal cell include early promoter and enhancer of SV40 (Mizukami and Itoh, 1987), LTR promoter and enhancer of Moloney mouse leukemia virus (Kuwana et al., 1987), promoter (Mason et al., 1985) and enhancer (Gillies et al., 1983) of immunoglobulin H chain and the like.

A further aspect of the present invention relates to a host cell genetically transformed with a nucleic acid molecule of the present invention. In particular, the host cell is a prokaryotic or eukaryotic host cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”. In some embodiments, for expressing and producing the polypeptide of the present invention, prokaryotic cells, in particular E. coli cells, will be chosen. Actually, according to the invention, it is not mandatory to produce the polypeptide of the present invention in a eukaryotic context that will favour post-translational modifications (e.g. glycosylation). Furthermore, prokaryotic cells have the advantages to produce protein in large amounts. If a eukaryotic context is needed, yeasts (e.g. saccharomyces strains) may be particularly suitable since they allow production of large amounts of proteins. Otherwise, typical eukaryotic cell lines such as CHO, BHK-21, COS-7, C127, PER.C6, YB2/0 or HEK293 could be used, for their ability to process to the right post-translational modifications of the polypeptide of the present invention. The construction of expression vectors in accordance with the invention, and the transformation of the host cells can be carried out using conventional molecular biology techniques. The polypeptide of the present invention, can, for example, be obtained by culturing genetically transformed cells in accordance with the invention and recovering the polypeptide expressed by said cell, from the culture. They may then, if necessary, be purified by conventional procedures, known in themselves to those skilled in the art, for example by fractional precipitation, in particular ammonium sulfate precipitation, electrophoresis, gel filtration, affinity chromatography, etc. In particular, conventional methods for preparing and purifying recombinant proteins may be used for producing the proteins in accordance with the invention. Thus a further aspect of the present invention relates to a method for producing a polypeptide of the present invention comprising the step consisting of: (i) culturing a transformed host cell according to the invention under conditions suitable to allow expression of said polypeptide; and (ii) recovering the expressed polypeptide.

In some embodiments, it is contemplated that the polypeptide of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. For instance, a strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. For example, Pegylation is a well-established and validated approach for the modification of a range of polypeptides (Chapman, 2002). The benefits include among others: (a) markedly improved circulating half-lives in vivo due to either evasion of renal clearance as a result of the polymer increasing the apparent size of the molecule to above the glomerular filtration limit, and/or through evasion of cellular clearance mechanisms; (b) reduced antigenicity and immunogenicity of the molecule to which PEG is attached; (c) improved pharmacokinetics; (d) enhanced proteolytic resistance of the conjugated protein (Cunningham-Rundles et. al., 1992); and (e) improved thermal and mechanical stability of the PEGylated polypeptide. Therefore, in some embodiments, the polypeptides of the present invention may be covalently linked with one or more polyethylene glycol (PEG) group(s).

In some embodiments, the polypeptide of the present invention is conjugated to a ligand, such as biotin (e.g., via a cysteine or lysine residue), a lipid molecule (e.g., via a cysteine residue), or a carrier protein (e.g., serum albumin, immunoglobulin Fc domain via e.g., a cysteine or lysine residue). Attachment to ligands, such as biotin, can be useful for associating the peptide with ligand receptors, such as avidin, streptavidin, polymeric streptavidin (see e.g., US 2010/0081125 and US 2010/0267166, both of which are herein incorporated by reference), or neutravidin. Avidin, streptavidin, polymeric streptavidin, neutravidin, in turn, can be linked to a signaling moiety (e.g., a moiety that can be visualized, such as colloidal gold, a fluorescent moiety, or an enzyme (horseradish peroxidase or alkaline phosphatase) or a solid substrate (e.g., an Immobilon or nitrocellulose membrane). Alternatively, the polypeptide of the present invention can be fused or linked to a ligand receptor, such as avidin, streptavidin, polymeric streptavidin, or neutravidin, thereby facilitating the association of the peptides with the corresponding ligand, such as biotin and any moiety (e.g., signaling moiety) or solid substrate attached thereto. Examples of other ligand-receptor pairs are well-known in the art and can similarly be used.

In some embodiments, the polypeptide of the present invention is fused to a fusion partner (e.g., a peptide or other moiety) that can be used to improve purification, to enhance expression of the peptide in a host cell, to aid in detection, to stabilize the peptide, etc. Examples of suitable compounds for fusion partners include carrier proteins (e.g., serum albumin, immunoglobulin Fc domain), beta-galactosidase, glutathione-S-transferase, a histidine tag, etc. The fusion can be achieved by means of, e.g., a peptide bond. A further object of the present invention relates to a method of treating C3 NeF associated C3 Glomerulopathy, Dense Deposit Disease (DDD) or Barraquer-Simons syndrome in a subject in thereof comprising administering to the subject a therapeutically effective amount of a polypeptide or nucleic acid molecule of the present invention.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

By a “therapeutically effective amount” is meant a sufficient amount of the polypeptide or nucleic acid molecule of the present invention for reaching a therapeutic effect (e.g. treating C3 NeF associated C3 Glomerulopathy). It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Another object of the present invention relates to a pharmaceutical composition comprising the polypeptide or nucleic acid molecule of the present invention and a pharmaceutically acceptable carrier. Typically, the polypeptide or the nucleic acid molecule can be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to the subjects. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the present invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide or nucleic acid molecule of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The polypeptides of the present invention are also suitable for detection the presence of C3 NeF in a sample. Thus a further object of the present invention relates to a method for detecting the presence of C3 Nef autoantibodies in a sample comprising contacting the sample with the polypeptide of the present invention under conditions that allow an immunocomplex of the polypeptide and the antibody to form, and detecting the presence of the immunocomplex.

Detection of the immunocomplexes indicates that the subject suffers from C3 NeF associated C3 Glomerulopathy or Barraquer-Simons syndrome.

In some embodiments, the sample is blood sample. As used herein, the term “blood sample” refers to a whole blood sample, serum sample and plasma sample. A blood sample may be obtained by methods known in the art including venipuncture or a finger stick. Serum and plasma samples may be obtained by centrifugation methods known in the art. The sample may be diluted with a suitable buffer before conducting the assay.

Assays and conditions for the detection of immunocomplexes are known to those of skill in the art. Such assays include, for example, competition assays, direct reaction assays sandwich-type assays and immunoassays (e.g. ELISA). The assays may be quantitative or qualitative. There are a number of different conventional assays for detecting formation of an antibody-peptide complex comprising a polypeptide of the present invention. For example, the detecting step can comprise performing an ELISA assay, performing a lateral flow immunoassay, performing an agglutination assay, analyzing the sample in an analytical rotor, or analyzing the sample with an electrochemical, optical, or opto-electronic sensor. These different assays are well-known to those skilled in the art.

In some embodiments, the assay utilizes a solid phase or substrate to which the polypeptide of the present invention is directly or indirectly attached. Accordingly in some embodiments, the polypeptide of the present invention is attached to or immobilized on a substrate, such as a solid or semi-solid support. The attachment can be covalent or non-covalent, and can be facilitated by a moiety associated with the polypeptide that enables covalent or non-covalent binding, such as a moiety that has a high affinity to a component attached to the carrier, support or surface. For example, the polypeptide can be associated with a ligand, such as biotin, and the component associated with the surface can be a corresponding ligand receptor, such as avidin. The polypeptide can be attached to or immobilized on the substrate either prior to or after the addition of the sample during an immunoassay. In some embodiments, the substrate is a bead, such as a colloidal particle (e.g., a colloidal nanoparticle made from gold, silver, platinum, copper, metal composites, other soft metals, core-shell structure particles, or hollow gold nanospheres) or other type of particle (e.g., a magnetic bead or a particle or nanoparticle comprising silica, latex, polystyrene, polycarbonate, polyacrylate, or PVDF). Such particles can comprise a label (e.g., a colorimetric, chemiluminescent, or fluorescent label) and can be useful for visualizing the location of the polypeptides during immunoassays. In some embodiments, a terminal cysteine of a polypeptide of the invention is used to bind the peptide directly to the nanoparticles made from gold, silver, platinum, copper, metal composites, other soft metals, etc. In some embodiments, the substrate is a dot blot or a flow path in a lateral flow immunoassay device. For example, the polypeptides can be attached or immobilized on a porous membrane, such as a PVDF membrane (e.g., an Immobilon™ membrane), a nitrocellulose membrane, polyethylene membrane, nylon membrane, or a similar type of membrane. In some embodiments, the substrate is a flow path in an analytical rotor. In some embodiments, the substrate is a tube or a well, such as a well in a plate (e.g., a microtiter plate) suitable for use in an ELISA assay. Such substrates can comprise glass, cellulose-based materials, thermoplastic polymers, such as polyethylene, polypropylene, or polyester, sintered structures composed of particulate materials (e.g., glass or various thermoplastic polymers), or cast membrane film composed of nitrocellulose, nylon, polysulfone, or the like. A substrate can be sintered, fine particles of polyethylene, commonly known as porous polyethylene, for example, 0.2-15 micron porous polyethylene from Chromex Corporation (Albuquerque, N. Mex.). All of these substrate materials can be used in suitable shapes, such as films, sheets, or plates, or they may be coated onto or bonded or laminated to appropriate inert carriers, such as paper, glass, plastic films, or fabrics. Suitable methods for immobilizing peptides on solid phases include ionic, hydrophobic, covalent interactions and the like.

Accordingly, in another aspect, the invention provides devices. In certain embodiments, the devices are useful for performing an immunoassay according to the present invention. For example, in some embodiments, the device is a lateral flow immunoassay device. In some embodiments, the device is an analytical rotor. In some embodiments, the device is a dot blot. In some embodiments, the device is a tube or a well, e.g., in a plate suitable for an ELISA assay. In some embodiments, the device is an electrochemical sensor, an optical sensor, or an opto-electronic sensor.

The presence and amount of the immunocomplex may be detected by methods known in the art, including label-based and label-free detection. For example, label-based detection methods include addition of a secondary antibody that is coupled to an indicator reagent comprising a signal generating compound. The secondary antibody may be an anti-human IgG antibody. Indicator reagents include chromogenic agents, catalysts such as enzyme conjugates, fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors and magnetic particles. Examples of enzyme conjugates include alkaline phosphatase, horseradish peroxidase and beta-galactosidase. Methods of label-free detection include surface plasmon resonance, carbon nanotubes and nanowires, and interferometry. Label-based and label-free detection methods are known in the art and disclosed, for example, by Hall et al. (2007) and by Ray et al. (2010) Proteomics 10:731-748. Detection may be accomplished by scanning methods known in the art and appropriate for the label used, and associated analytical software. In some embodiments, fluorescence labeling and detection methods are used to detect the immunocomplexes.

In some embodiments of the invention, the peptide is provided with a suitable label which enables detection. Conventional labels may be used which are capable, alone or in concert with other compositions or compounds, of providing a detectable signal. Suitable detection methods include, e.g., detection of an agent which is tagged, directly or indirectly, with a fluorescent label by immunofluorescence microscopy, including confocal microscopy, or by flow cytometry (FACS), detection of a radioactively labeled agent by autoradiography, electron microscopy, immunostaining, subcellular fractionation, or the like. In one embodiment, a radioactive element (e.g., a radioactive amino acid) is incorporated directly into a peptide chain; in another embodiment, a fluorescent label is associated with a peptide via biotin/avidin interaction, association with a fluorescein conjugated antibody, or the like. In one embodiment, a detectable specific binding partner for the antibody is added to the mixture. For example, the binding partner can be a detectable secondary antibody or other binding agent (e.g., protein A, protein G, protein L) which binds to the first antibody. This secondary antibody or other binding agent can be labeled, e.g., with a radioactive, enzymatic, fluorescent, luminescent, or other detectable label, such as an avidin/biotin system. In another embodiment, the binding partner is a peptide of the invention, which can be conjugated directly or indirectly (e.g. via biotin/avidin interaction) to an enzyme, such as horseradish peroxidase or alkaline phosphatase. In such embodiments, the detectable signal is produced by adding a substrate of the enzyme that produces a detectable signal, such as a chromogenic, fluorogenic, or chemiluminescent substrate.

In some embodiments, the detection procedure comprises visibly inspecting the antibody-polypeptide complex for a color change, or inspecting the antibody-polypeptide complex for a physical-chemical change. Physical-chemical changes may occur with oxidation reactions or other chemical reactions. They may be detected by eye, using a spectrophotometer, or the like.

A particularly useful assay format is a lateral flow immunoassay format. Antibodies to human or animal (e.g., dog, mouse, deer, etc.) immunoglobulins, or staph A or G protein antibodies, can be labeled with a signal generator or reporter (e.g., colloidal gold) that is dried and placed on a glass fiber pad (sample application pad or conjugate pad). The diagnostic polypeptide is immobilized on membrane, such as nitrocellulose or a PVDF (polyvinylidene fluoride) membrane (e.g., an Immobilon™ membrane). When a solution of sample (blood, serum, etc.) is applied to the sample application pad (or flows through the conjugate pad), it dissolves the labeled reporter, which then binds to all antibodies in the sample. The resulting complexes are then transported into the next membrane (PVDF or nitrocellulose containing the diagnostic polypeptide of the present invention) by capillary action. If antibodies against the diagnostic polypeptide are present, they bind to the diagnostic polypeptide striped on the membrane, thereby generating a signal (e.g., a band that can be seen or visualized). An additional antibody specific to the labeled antibody or a second labeled antibody can be used to produce a control signal. An alternative format for the lateral flow immunoassay comprises the polypeptides of the present invention being conjugated to a ligand (e.g., biotin) and complexed with labeled ligand receptor (e.g., streptavidin-colloidal gold). The labeled polypeptide complexes can be placed on the sample application pad or conjugate pad. Anti-human IgG/IgM or anti-animal (e.g., dog, mouse, deer) IgG/IgM antibodies or other polypeptides of the invention are immobilized on a membrane, such as nitrocellulose of PVDF, at a test site (e.g., a test line). When sample is added to the sample application pad, antibodies in the sample react with the labeled polypeptide complexes such that antibodies that bind to polypeptides of the invention become indirectly labeled. The antibodies in the sample are then transported into the next membrane (PVDF or nitrocellulose containing the diagnostic polypeptide) by capillary action and bind to the immobilized anti-human IgG/IgM or anti-animal IgG/IgM antibodies (or protein A, protein G, protein L, or combinations thereof) or immobilized polypeptides of the invention. If any of the sample antibodies are bound to the labeled polypeptides of the invention, the label associated with the peptides can be seen or visualized at the test site.

Another assay is an enzyme linked immunosorbent assay, i.e., an ELISA. Typically in an ELISA, isolated polypeptides of the present invention are adsorbed to the surface of a microtiter well directly or through a capture matrix (e.g., an antibody). Residual, non-specific protein-binding sites on the surface are then blocked with an appropriate agent, such as bovine serum albumin (BSA), heat-inactivated normal goat serum (NGS), or BLOTTO (a buffered solution of non-fat dry milk which also contains a preservative, salts, and an antifoaming agent). The well is then incubated with the sample. The sample can be applied neat, or more often it can be diluted, usually in a buffered solution which contains a small amount (0.1-5.0% by weight) of protein, such as BSA, NGS, or BLOTTO. After incubating for a sufficient length of time to allow specific binding to occur, the well is washed to remove unbound protein and then incubated with an optimal concentration of an appropriate anti-immunoglobulin antibody (e.g., for human subjects, an anti-human immunoglobulin (αHulg) from another animal, such as dog, mouse, cow, etc. that is conjugated to an enzyme or other label by standard procedures and is dissolved in blocking buffer. The label can be chosen from a variety of enzymes, including horseradish peroxidase (HRP), beta-galactosidase, alkaline phosphatase, glucose oxidase, etc. Sufficient time is allowed for specific binding to occur again, then the well is washed again to remove unbound conjugate, and a suitable substrate for the enzyme is added. Color is allowed to develop and the optical density of the contents of the well is determined visually or instrumentally (measured at an appropriate wave length).

A further object of the present invention relates to a kit for detecting C3Nef in a sample. The kit comprises one or more polypeptides of the present invention and means for determining binding of the polypeptides to C3Nef in the sample. Reagents for particular types of assays can also be provided in kits of the invention. Thus, the kits can include a population of beads (e.g., suitable for an agglutination assay or a lateral flow assay), or a plate (e.g., a plate suitable for an ELISA assay). In some embodiments, the kits comprise a device, such as a lateral flow immunoassay device, an analytical rotor, or an electrochemical, optical, or opto-electronic sensor. The population of beads, the plate, and the devices are useful for performing an immunoassay. In addition, the kits can include various diluents and buffers, labeled conjugates or other agents for the detection of the specifically immunocomplexes, and other signal-generating reagents, such as enzyme substrates, cofactors and chromogens. Other components of a kit can easily be determined by one of skill in the art. The kits are useful for diagnosing C3 NeF associated C3 Glomerulopathy or Barraquer-Simons syndrome.

The invention will be further illustrated by the following figures and examples. However, these examples and figures should not be interpreted in any way as limiting the scope of the present invention.

### FIGURES

FIG. 1: Peptide inhibition assay. (A) % of inhibition of peptide 1, 2 and 3 pre-incubated with Properdin. The % of inhibition was calculated as the % of residual convertase of the Properdin-peptides mix compared to that of Properdin-peptide's excipient. (B) % of inhibition in presence of different doses of peptides (1 in blue, 2 in red and 3 in orange). (C) % of inhibition of peptides pre-incubated with P1 IgG. (D) % of inhibition in presence of different doses of peptide 1. (E) % of inhibition of peptide 1 on the stabilization of 3 C3NeFs stabilizing only C3bBb and 3 C3NeFs stabilizing both convertases. Statistic tests are made by 1 way ANOVA Kruskal-Wallis test, * correspond to p<0.05.

FIG. 2: Peptides reproducing conformational epitopes on the C3 convertase and containing sequences from the C345C domain of C3 and the vWF type A domain of FB.

FIG. 3: Competition assays between properdin and peptides. C3bBb membrane bound convertase was formed on C3b erythrocytes and Peptides and properdin were added during the convertase decay. Competition between properdin and 4 peptides are showed. Panel A: Direct binding of Properding to peptides. Panel B: Functional inhibition.

FIG. 4: Peptide inhibition assay. % of inhibition of peptides pre-incubated with patient' IgG.

### EXAMPLE 1

Methods

Patients

Between 2001 and 2013, 149 patients were included in the French C3 glomerulopathy registry and EDTA blood samples were sent to the laboratory of Immunology (Hôpital Européen Georges Pompidou, Paris, France) for complement assessment. The inclusion criteria were adapted to the recent classification of C3 glomerulopathy. All kidneys biopsy reports were reviewed and patients were selected according to the identification of the immune reactant by immunofluorescence study. Patients were included if they demonstrated the presence of glomerular staining of C3 with at least two order of magnitude of intensity for others immune reactants by immunofluorescence. In France, the electron microscopy is not performed routinely for GN characterisation and thus EM analysis of biopsy was missing in 90% of cases. Therefore the electron-dense appearance of deposits in glomeruli, which correspond to the C3 detected by IF was unavailable. Using light microscopy morphological features and Immunofluorescence of a biopsy sample pathologists recognized 44 cases of Dense Deposits diseases and 77 cases of C3 glomerulonephritis with predominant C3 deposits.

We excluded for this study patients with monoclonal gammopathy detected by immunofixation (n=28 patients) and patients with identified mutations in Factor H (n=12), Factor I (n=3), MCP (n=1), C3 (n=5) and CFHR 5 (n=1). Two out the 20 patients carried combined mutations in C3 and Factor I. None of the patients carry a mutation in Factor B gene. We also excluded 12 patients in whom complement assessment had been performed during the time of transplantation to avoid the influence of immunosuppressive drug on the antibody titres. Plasma samples were available for 89 patients for C3NeF screening and for assessment of complement biomarkers. For 36/89 samples insufficient volume of plasma or IgG available prevented us from performing of haemolytic assay with Properdin (data not shown). None of the patients presented anti-Properdin and anti-FB antibodies.

Genetic Analysis and Complement Plasma Proteins Levels

The DNA was extracted from whole blood using proteinase K/phenol method (Dragon-Durey et al. JASN 2003) for direct sequencing of all CFH, CFI, MCP, C3, FB and CFHR5 exons. EDTA plasma samples were used for measurement of C3, FH and FI concentrations as previously described (Roumenina et al. J Immunol Methods 2011). Soluble C5b9 level determination was done using the MicroVue sC5b-9 Plus EIA Assay (Quidel, San Diego, Calif.), according to manufacturer instructions. Normal values were evaluated testing plasma from 50 healthy donors.

Assays for C3NeF Detection.

The ability of patient' IgG to stabilize a pre-formed membrane bound C3bBb was measured with EAC3bBb generated by the incubation of EC3b with appropriate concentration of FB and FD. Briefly, IgG was partially purified from plasma by chromatography on DEAE-cellulose and subsequently quantified by nephelometry. DEAE eluate contains IgG, and also low levels of contaminants such as Properdin and Factor H. Sheep erythrocytes EAC34b were incubated in presence of FB (Complement Technology) and FD (Sigma) to form the alternative convertase C3bBb. The convertase was allowed to decay in EDTA-containing buffer in presence of 400 μg of patient' IgG. Lysis was developed by the addition of rat serum. C3NeF stabilization was measured as the percentage of the residual convertase compared to the reference control without decay. The cut-off for positive C3NeF was determined as the mean+2 sd of the C3NeF stabilization of IgG from 30 healthy individuals.

The ability of patient' IgG to stabilize a pre-formed membrane bound C3bBbP was measured with EAC3bBb generated by the incubation of EAC3b with appropriate concentrations of FB FD and Properdin (Calbiochem). This assay is similar to the previous one, but the C3bBbP convertase was let decay for 30 minutes at 30° C. The cut-off for positive C3NeF was determined as the mean+2 sd of the C3NeF stabilization of IgG from 30 healthy individuals.

Assessment of C3NeF Binding to the Convertase by Surface Plasmon Resonance (SPR)

C3NeF binding to the convertase was studied by surface plasmon resonance (SPR) with ProteOn XPR36 equipment (BioRad). C3b was coupled to the GLC biosensor chip and C3 convertases C3bBb and C3bBbP were formed by flowing on chip respectively FB (3 μg/ml) and FD (0.3 μg/ml) or FB (1 μg/ml), FD (0.1 μ/ml) and FP (0.5 μg/ml) in a Mg²⁺ containing buffer (10 mM Hepes pH 7.4, 40 mM NaCl, 10 mM MgCl₂, 0.005% surfactant P20). After convertase formation, 100 ng of purified C3NeF positive IgGs or control IgG from healthy donors were injected over the convertase containing flow cell and one flow cell in which buffer was flowed instead of FB, FD and/or FP, used as control for background bindings to coated C3b. The association of IgG to the convertase and the dissociation of the complex were followed in real-time for 300 seconds and 600 seconds respectively. Data were analyzed using ProteOn Manager Software and the data from the control flow cell were subtracted.

Competition Assay

Membrane-bound convertase was formed as in the C3bBb assay. During the convertase decay step, cells were incubated with FP alone at a fixed dose (800 ng); 4 different doses of IgG alone and a mix of both IgG and FP at the same concentration. The maximal concentration of IgG applied in the test was chosen depending on their C3NeF activity. Similarly, the competition was made with a fixed amount of IgG and different doses of FP.

Peptides Design

3 peptides, reproducing conformational neo-epitopes on the C3 convertase C3bBb and containing sequences from the C345C domain of C3 and the vWF type A domain of FB were designed by mapping the surface areas, spanning the putative Properdin binding site. The Properdin binding area was deduced from the previously published electron microscopy images (Alcorlo et al., PNAS, 2013). Different peptides were selected manually by visualization of the surface area of the C3bBb complex with SCIN (PDB ID 2WIN), after removal of the sequence corresponding to SCIN. Peptide 1 covered 22 residues of C3b (1616-1637) and 11 residues of Bb (308-318) (G-QDEENQKQCQDLGAFTESMVVFNIENLQKTVWD) (SEQ ID NO:5). Peptide 2 consisted of 3 parts of C3b (1609-1614; 1544-1547; 1635-1636) and of one part of the vWFA domain of Bb (292-297) (G-EDEEPWEQITVV YPKIWV) (SEQ ID NO: 8). Peptide 3 included 9 adjacent residues (1508-1516) and 1 detached residue (1641) of C3b and 10 adjacent residues (254-263) and 1 detached residue (368) of Bb (G-EERLDKACENNDSIGASNFTG) (SEQ ID NO: 9). A glycine was added at the beginning of each peptide to avoid the presence of problematic aminoacids at the N-terminus. Peptides were synthesized by ThermoFisher Scientific with a purity of >90% and re-suspended in PBS/DMSO 5% in order to obtain a final concentration of 1 mg/ml.

Peptides Inhibition Assay

Different mixes of 50 ng of Properdin and 10 μg of peptides were incubated at room temperature for 2 hours with gentle shaking in EDTA-containing buffer. As a control, Properdin was incubated with the peptide's dilution buffer. Membrane-bound convertase was formed as in the C3bBb assay. During the convertase decay step, cells were incubated with the mix of FP and peptides. FP amount was chosen in order to obtain 50% of residual convertase after the decay. Residual convertase was measured and data were expressed as percentage of inhibition (percentage of residual convertase compared to the control without peptide).

The test was performed as described earlier, but 80 μg of peptides was utilized and the incubation of IgG and peptides was made at 4° C. overnight. IgG amount was determined depending on their stabilization capacity, in order to obtain 50% of residual convertase after the decay.

Results

Identification of Patients with C3NeF Using the C3b-Coated Erythrocytes, Based on Alternative Pathway C3 Convertase Stabilization Assays

To evaluate the ability of C3Nef to stabilize the C3 convertase, we used a fixed concentration of 400 μg patient or control IgG and determined the residual number of lytic sites per cell (Z) after 20 minutes of incubation with EAC3bBb cells with the IgG preparations. The results were calculated and expressed as percent of lytic sites per cell (Z) corresponding to the % of C3bBb stabilization. In presence of the same amount of IgG prepared from healthy donors the residual convertase at 20 minutes was less than 20% of convertase generated at the initial convertase. Of the 89 patients with C3G, 69 (78%) were positive for C3NeF in this assay. The percentage of residual lytic sites varies from 20 to 100% of the initial convertase. The % of stabilization was between 20% to 40%, 40% to 80% and 80% to 100% for 44% (n=30), 36% (n=25) and 20% (n=14) respectively (data not shown). The stabilization of the C3 convertase is IgG dose dependant and 50% of stabilization is observed with concentration of 1 μg to 400 μg of total IgG in the assay depending on the patient tested (data not shown).

Identification of Patients with C3NeF Using C3b-Coated Erythrocytes Based Assay of Properdin-Stabilized Alternative Pathway C3 Convertase

To evaluate the ability of C3NeF to bind to and stabilize the C3 convertase in presence of Properdin we developed a new haemolytic test. We used a fixed amount of patient or control IgG (400 μg) to determine the residual lytic sites after 30 minutes of incubation of the IgG preparations with EAC3bBbP cells. Less than 20% of lytic sites per cell was observed at 30 minutes with the same amount of IgG prepared from healthy donors (n=30).

Of the 55 tested patients, 28 (51%) were positive for C3NeF in this assay. The percentage of residual lytic sites here varies from 20 to 100% of the initial convertase. The % of stabilization was between 20% and 40%, 40% to 80% and 80% to 100% for 22% (n=6), 39% (n=11) and 39% (n=11) respectively (data not shown). The capacity of C3NeF to stabilize the C3bBbP is IgG dose dependant.

Of the 55 tested samples, 44 (80%) were positive in C3bBb and/or C3bBbP stabilization assay (data not shown). Patients' IgG stabilized both the C3bBb and the C3bBbP convertases in 50% of positive C3NeF samples (n=22). C3bBb stabilization convertase assays detected C3NeF which were negative in C3bBbP stabilization convertase assays (36% of positive C3NeF, n=16). C3bBbP stabilization convertase assays detected C3NeF which were negative in C3bBb stabilization convertase assays (14% of positive C3NeF, n=6).

Analysis of C3NeF Binding to the Convertase by Surface Plasmon Resonance

C3NeF positive IgG were tested for their capacity to bind to the convertases C3bBb and C3bBbP in real time by SPR. Tested IgG were representative of the range of characteristics observed in C3NeF functional assay (2 patients' IgG positive in the C3bBb based C3NeF haemolytic assay (P1, P2) and 3 patients' IgG positive in both of the assays (P3, P4, P5). IgG from three healthy donors were used as controls.

C3b was immobilized on chip and convertase was formed by flowing FD and FB in one case and FD, FB and Properdin in another case. After convertases formation, IgG were flowed on the chip and their binding to the complex was followed. Binding signal was increased when P1 and P2 IgG were flowed in the presence of C3bBb but no binding signal was observed in the presence of C3bBbP convertase (data not shown). Binding signal was increased when P3, P4 and P5 IgG were flowed on both the C3bBb and on the C3bBbP convertases (data not shown).

Biomarkers of Complement Activation in C3G Patients According to the C3Nef Status

To evaluate the influence of the presence of different types of C3Nef on the level of complement activation in patients, we measured C3 and sC5b9 plasmatic levels. C3 levels were significantly lower in patients with C3NeF compared to patients without. The C3 levels were below the normal range (<660 mg/ml) for 52% of patients with C3Nef against the C3bBb (including 11 (16%) patients with C3 level below 200 mg/ml), and for 25% of patients with no detectable C3NeF (data not shown). The concentration of sC5b9 was not significantly different between the groups of patients with and without C3NeF against the C3bBb (data not shown).

In the case of C3NeF stabilizing C3bBbP, 41% of patients with C3NeF had significantly lower C3 levels compared to patients without (data not shown). Contrary to the previous one, in presence of C3NeF against C3bBbP, levels of sC5b9 were strongly increased (data not shown).

C3 levels were significantly lower in patients with C3NeF against both the C3bBb and the C3bBbP convertases or only C3bBbP convertase when compared to C3NeF negative patients (data not shown). The sC5b9 was significantly higher in the groups of C3NeF directed against the C3bBbP or C3bBb and C3bBbP than in the group of C3Nef against the C3bBb alone (data not shown).

Characterisation of the Binding Area of C3NeF

We raised the possibility that the area of the C3Nef binding site drive the functional consequences on the complement activation. Since both C3Nef and Properdin stabilize the C3 convertase and because we found C3Nef samples which stabilize the C3bBb convertase and have no effect on the C3bBbP convertase stabilization, we hypothesized that the area of C3NeFs binding site is located within the Properdin binding site in the C3bBb convertase (Properdin-like C3NeF). However C3NeF which stabilize the C3bBb and C3bBbP convertases should bind the surface of the C3 convertase at a different site compared to Properdin. In addition, this binding area should not be affected by the conformational change induce by the binding of Properdin to the C3 convertase (Properdin Independent C3NeF). Lastly we proposed that C3NeF which stabilize the C3bBbP convertase and has no effect on the C3bBb convertase stabilization is dependent on the presence of Properdin on the C3 convertase (Properdin Dependent C3NeF).

To test the hypothesis that C3NeF would share the same epitope as Properdin, we performed a competitive assay between patient IgG and C3NeF. In the C3b Erythrocyte based convertase stabilization assays, 60% of residual C3bBb lytic site is obtained with 800 ng of Properdin. We used four representative C3NeF samples which stabilize only the C3bBb (n=2, P1 and P2) and both the C3bBb and C3bBbP (n=2, P3 and P4).

Patients' IgG (P1, P2, P3 and P4) added at the time of dissociation step increase the percentage of stabilization in a dose dependent manner. 50, 300, 150 and 300 microG of IgG induce 50% of convertase stabilisation for P1, P2, P3 and P4 IgG respectively. Properdin added at the same time of P3 and P4′ IgG increases the percentage of stabilization in a IgG dose dependant manner and rich a maximum of stabilization with 20 microG of IgG (data not shown). Our data show that Properdin and C3NeF cooperate in stabilizing the C3 convertase.

At opposite, increasing amount of P1 and P2 IgG induce a decrease % of stabilisation of fixed amount of Properdin (data not shown). Moreover, in presence of P1 IgG the % of stabilisation doesn't change whatever the concentration of Properdin added in the assay (data not shown). Our data show that Properdin and C3NeF compete in stabilizing the C3 convertase.

Identification of C3NeF and Properdin Binding Site

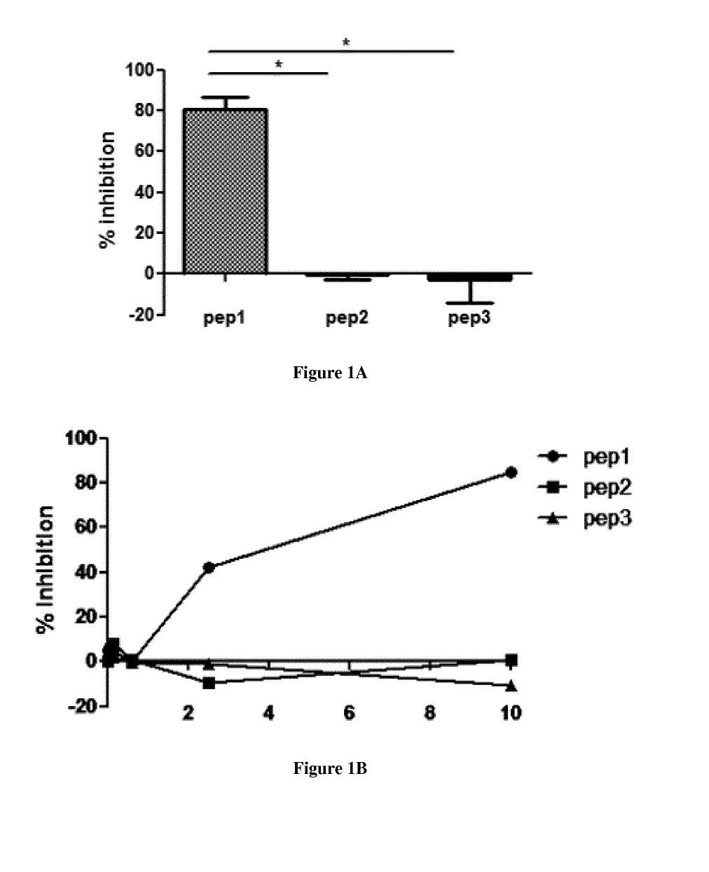

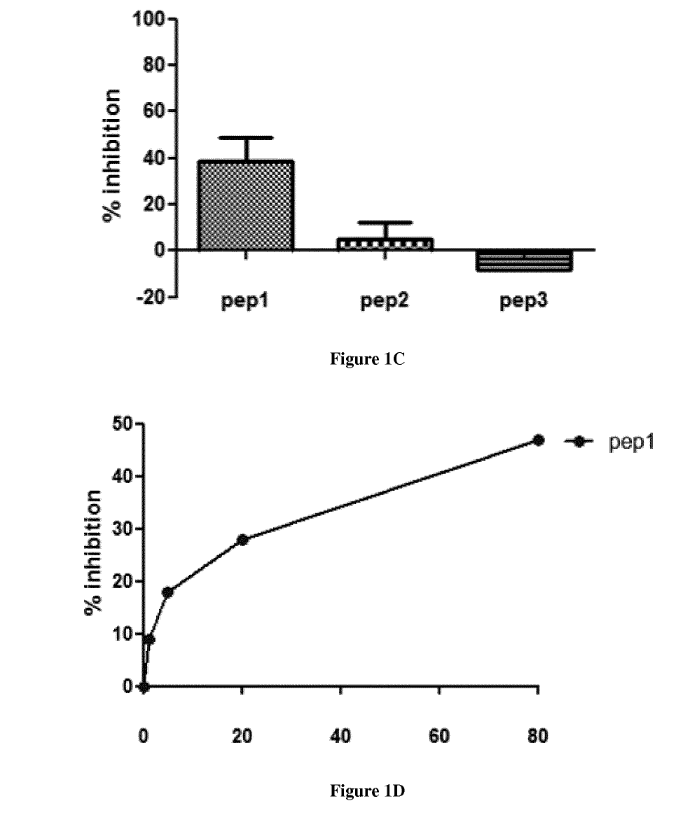

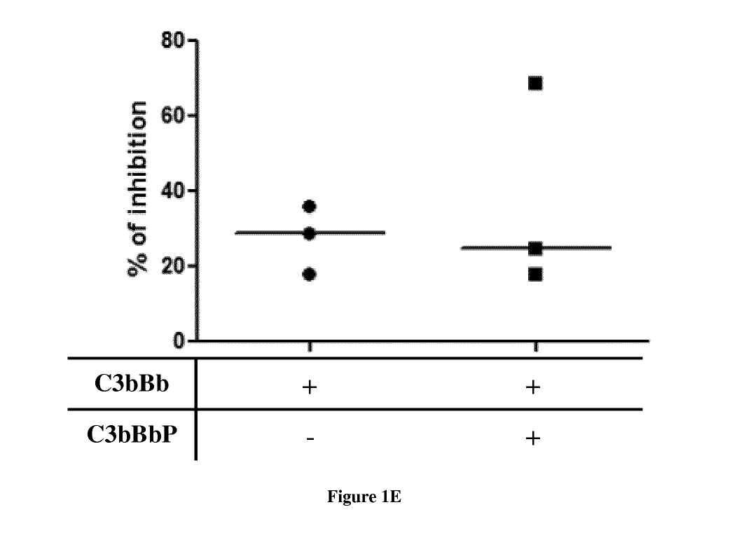

To better define the binding site of Properdin on the C3 convertase, we designed 3 different peptides: one reproducing the putative Properdin binding site (peptide 1) as deduced by the pseudo atomic structure proposed by Alcorlo et al.; two reproducing regions on the C3 convertase on the right (peptide 2) and on the left (peptide 3) side (data not shown). We tested the ability of these peptides to inhibit the stabilization of the C3 convertase mediated by Properdin (peptide inhibition assay). Properdin was pre-incubated with the peptides and this mixture was added at the time of convertase dissociation step. Properdin incubated with peptides' excipient reached 40% of stabilization. In presence of peptide 1 this effect was almost completely abrogated, reaching 80% inhibition of the Properdin stabilization capacity (n=2 experiments). The inhibition was dose-dependent. Peptide 2 and 3 had no effect (FIGS. 1A and 1B).

Having mapped the putative Properdin binding site on C3bBb to the sequence corresponding to Peptide 1, we tested the capacity of the peptides to reduce also C3NeF stabilization. We firstly screened C3NeF sample from P1, able to stabilize only C3bBb. IgG from P1 and peptides were pre-incubated overnight and then added during the convertase decay step. As for Properdin, we compared stabilization of IgG alone and of the IgG-peptide complexes. The % stabilization of 100 μg of IgG (taken as 100% in absence of peptide) was decreased with 40% in the case of peptide 1 (n=3 experiments), but remained in the same range for peptides 2 and 3. Peptide 1 inhibited IgG stabilization in a dose dependent manner. (FIGS. 1C and 1D)

We tested peptide 1 inhibition also for other C3NeF samples: 3 which were positive for the C3bBb and negative for the C3bBbP test and 3, which are positive in both tests. In all cases C3NeF stabilization was diminished, with a range of 20-40% for the former group and of 20-70% for the latter. (FIG. 1E)

TABLE 1
IgG binding residual and K_{d} of convertase after IgG injection. IgG
binding on the convertases is calculated subtracting the median value
of RU of NHIgG to the RU of C3NeF positive taken at the end of the
IgG injection (at 900 seconds). K_{d} of the convertase were calculated
using ProteOn Manager Software by fitting sensorgrams into two state
interaction model.

| | |
|---|---|
| IgG binding, | |
| residual | |
| (RU) | K_{d} |

| | C3bBb | C3BbBP | C3bBb |
|---|---|---|---|
| P1 | 324 | 42 | 1.96E−04 |
| P2 | 107 | 67 | 7.62E−04 |
| P3 | 123 | 266 | 2.50E−03 |
| P4 | 217 | 122 | 1.83E−03 |
| P5 | 448 | 223 | 6.38E−04 |
| NHIgGsₘ | 1 | 13 | — |

### EXAMPLE 2

Therapeutic inhibition of C3 NeF using peptides may serve as promising treatment option in C3G. The inventors designed and extensively characterized three peptides that interfere with the Properdin and/or C3NeF in the stabilization of the C3 convertase. Since 1) both C3NeF and properdin stabilize the AP C3 convertase and 2) some C3NeF samples have no effect on the C3bBbP convertase stabilization, the inventors raised the hypothesis that the area of C3NeF binding site is located within the properdin binding site in the C3bBb convertase. Their first task is to identify the properdin binding site on C3b and on the C3bBb convertase using peptides inhibition assays. The binding area of Properdin on the convertase is unknown.

### A/ Peptides Design

### Strategy

The Properdin binding area was deduced from the previously published electron microscopy images. Different peptides were selected manually by visualization of the surface area of the C3bBb complex with SCIN (PDB ID 2WIN), after removal of the sequence corresponding to SCIN. To better define the binding site of Properdin on the C3 convertase, the inventors already designed 3 different peptides: one reproducing the putative Properdin binding site (peptide 1) as deduced by the pseudo atomic structure proposed by Alcorlo et al.; two reproducing regions on the C3 convertase on the right (peptide 2) and on the left (peptide 3) side. They tested the ability of these peptides to inhibit the stabilization of the C3 convertase mediated by Properdin (peptide inhibition assay). Properdin was pre-incubated with the peptides and this mixture was added at the time of convertase dissociation step. Properdin incubated with peptides' excipient reached 40% of stabilization. Peptide 1 covered 22 residues of C3b (1616-1637) and 11 residues of Bb (308-318) (G-QDEENQKQCQDLGAFTESMVVFNIENLQKTVWD). In presence of peptide 1 this effect was almost completely abrogated, reaching 80% inhibition of the Properdin stabilization capacity (n=2 experiments). The inhibition was dose-dependent. Peptide 2 and 3 had no effect.

### New Design

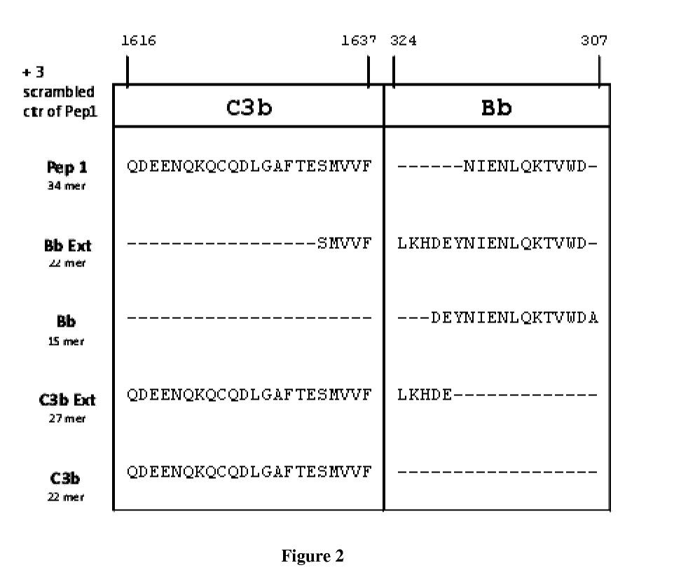

Four peptides, reproducing conformational neo-epitopes on the C3 convertase and containing sequences from the C345C domain of C3 and the vWF type A domain of FB were designed by mapping the surface areas, spanning the putative Properdin binding site. The peptides are depicted in FIG. 2. Using a peptide inhibition assay, the inventors tested the ability of ten newly design peptides to inhibit the stabilization of the C3 convertase mediated by Properdin. Peptides were synthesized by ThermoFisher Scientific with a purity of >90% and re-suspended in PBS/DMSO 5% in order to obtain a final concentration of 1 mg/ml. A glycine was added at the beginning of each peptide to avoid the presence of problematic aminoacids at the N-terminus. The inventors identified one polypeptide of 22 aminoacids comprising 5 aa on C3b and 17 from FB (BbExt) that inhibits the binding on Properdin to the C3 convertase.

### B/ Identification of Properdin Binding Site

### Methods:

### Epitope Competition Assay:

To investigate whether Properdin binds the peptides biotinylated peptides was coated onto ELISA plates and increased among of Properdin were incubated before adding anti human Properdin.

### Peptides Inhibition Assay

Different mixes of 50 ng of Properdin and 10 μg of peptides were incubated at room temperature for 2 hours with gentle shaking in EDTA-containing buffer. As a control, Properdin was incubated with the peptide's dilution buffer. Membrane-bound convertase was formed as in the C3bBb assay. During the convertase decay step, cells were incubated with the mix of FP and peptides. FP amount was chosen in order to obtain 50% of residual convertase after the decay. Residual convertase was measured and data were expressed as percentage of inhibition (percentage of residual convertase compared to the control without peptide).

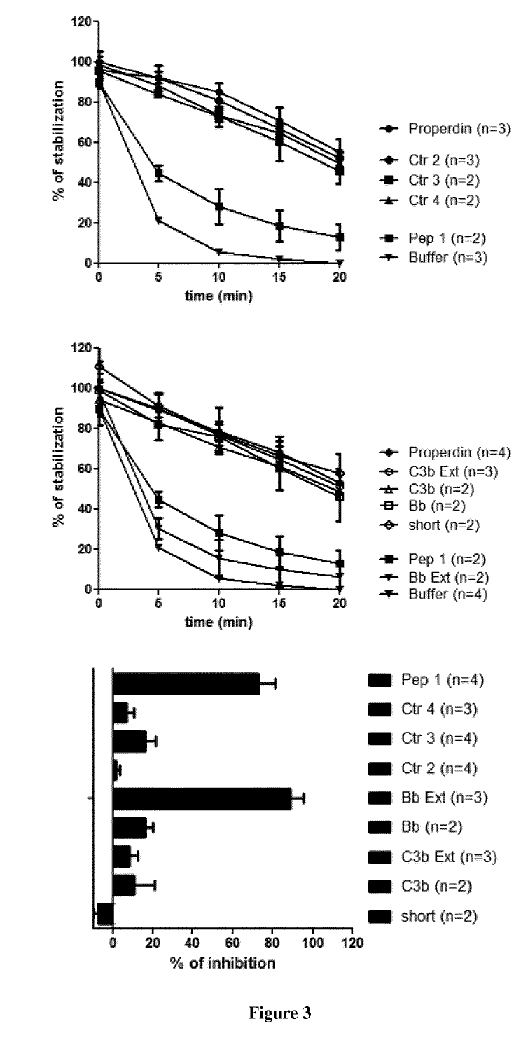

### Results: Characterisation of the Binding Area of Proderdin on the C3bBb Convertase (FIG. 3):

To better define the binding site of Properdin on the C3 convertase, the inventors tested the ability of these peptides to binds Properdin and to inhibit the stabilization of the C3 convertase mediated by Properdin. Properdin was pre-incubated with the peptides and this mixture was added at the time of convertase dissociation step. Properdin incubated with peptides' excipient reached 40% of stabilization. In presence of peptide 1 and BbExt this effect was almost completely abrogated, reaching 80% inhibition of the Properdin stabilization capacity (n=2 experiments). The inhibition was dose-dependent. Bb C3bExt and controls peptides had no effect. The ELISA measures the capacity of peptides to bind Properdin. The inventors found that BbExt and Pep1 binds the Properdin confirming the presence of a major epitope within the peptide derived from the C3bBb that interacts with Properdin. Importantly they identified two essential sequences for the binding of Properdin to the C3bBb convertases: NLQKTVWD on Bb and SMVVF on C3b.

### C/ Identification of C3NeF Epitopes Binding Site

The inventors use the term of C3NeF for all IgG capable to stabilize the C3 convertases C3bBb and C3bBbP. They identified two types of C3Nefs with different binding sites on the C3 convertase. C3NeF of one of the groups was found to bind at the Properdin binding site and to compete with it, sharing the same effect (Properdin independent C3NeF). The majority of C3NeF bind in an area, adjacent to the Properdin binding site and have an additive effect on the C3 convertase stabilization (Properdin enhancer C3 NeFs). Having mapped the putative Properdin binding site on C3bBb to the sequence corresponding to Peptide 1 and BbExt, but not Short and C3b Ext they tested the capacity of the peptides to reduce also C3NeF stabilization.

### Methods:

Membrane-bound convertase was formed as in the C3bBb assay. During the convertase decay step, cells were incubated with a fixed dose of Peptides (80 μg) and patient' IgG (amount determined by the concentration of IgG leading to 50% of residual convertase). The concentration of IgG applied in the test was chosen depending on their C3NeF activity.

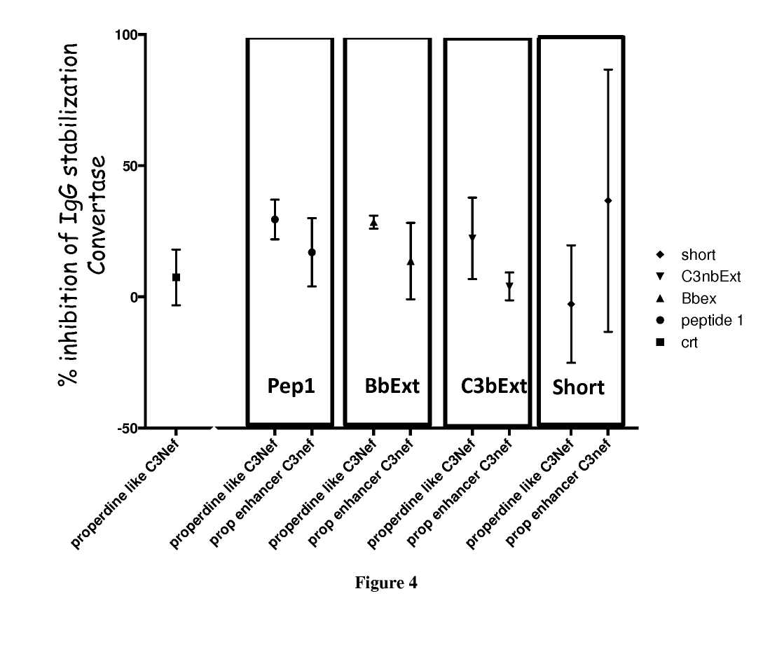

### Results (FIG. 4)

The inventors tested 3 patients' Ig which were positive for the C3bBb and negative for the C3bBbP test and 4, which are positive in both tests. In presence of 3 out 4 peptides the stabilization was diminished, with a range of 20-40% for the Properdin like C3NeF. At the opposite sole the peptides Short diminished the stabilization of the Convertase, with a range of 40-80% for the Properdin enhancer C3NeF. In presence of Properdin_like, the percentage of inhibition was significantly higher in presence of peptide1 vs control (p=0.005), of BbExt vs control (p=0.005), of C3bExt vs control (p=0.005). No significant difference was observed between control and short (short vs control (p=0.32). In presence of Properdin_enhancer, the percentage of inhibition was significantly higher in presence of short vs control (p=0.001). No significant difference was observed between control and Pep1, Bbext and C3bExt (p=0.25; 0.46; 0.61). Peptide inhibition experiments indicate this subtype of C3 NeF did not recognize the same area on the C3 convertase. These results confirm that one sub type of C3NeF and properdin, which both increase the AP activity, share the same binding site on the C3bBb convertase. By functional assays, the inventors demonstrated that three peptides inhibits the binding of patients' IgG on the C3 convertase to induce their functional effect. In cases of Properdin-enhancer C3 NeF the Peptides neutralizing Properdin binding will stop the amplification of the C3 convertase stabilization and limits the deleterious effect of the C5 activation. Lastly Peptides neutralizing Properdin binding will inhibit the Properdin-dependant C3 NeF binding in this context.

### CONCLUSION

C3NeF were found in approximately 65% of the patients of C3G which include the Dense Deposit Disease (DDD) and the C3 Glomerulonephritis (C3GN) and are associated with an overactivation of the alternative pathway (AP) of the complement. The antigen-driven expansion of self reactive B cell clones in response to a presence of C3 convertase in the kidney may explain the permanent production of C3 NeF and the echec of the Conventional Immunosuppresive therapy. The inventors propose to inhibit first the C3bBb target which will induce secondary the decrease of the polyclonal B cell response. For this, they proposed to target the C3bBb Convertase. Their results suggest that properdin participates to the uncontrolled activation of C3 and C5 convertases in patients with C3 NeF stabilizing the convertases with and without Properdin. Therefore therapeutic properdin inhibition (using antibodies or peptides) will have a benefit effect. At opposite, they demonstrated that C3 Nef autoantibodies which bind sole the C3bBb convertase have the same binding site with Properdin on the enzyme. Therefore specific therapeutic inhibition of properdin would be detrimental in this setting. The identification of different types of C3NeF with distinct functional specificities in vivo may have consequences for patients' management. Using the peptides therapeutic strategy, both Properdin and C3 NeF will not bind the Convertase. This inhibition of binding could induce secondary the disappearance of the C3bBb enzymes (decrease of the half life of the enzymes) and in the same time of the antigen-driven B cell clone producing the C3 NEf. These results show that inhibiting the binding of C3Nef and/Or Properdin to the enzyme will be a good strategy to treat Dense Deposit Disease (DDD), the C3 Glomerulonephritis (C3GN) and Barraquer Simons (partial lipodystrophy). More, the designed peptides could be used to design a new in vitro assay for the screening of C3 NeF.

### REFERENCES

Throughout this application, various references describe the state of the art to which this invention pertains. The disclosures of these references are hereby incorporated by reference into the present disclosure.

1. A polypeptide that is capable of inhibiting the binding of C3 NeF to C3 convertase and which comprises a first segment which consists of n consecutive amino acids selected in a first amino acid sequence set forth in SEQ ID NO:1 (GQDEENQKQCQDLGAFTESMVVF) fused to a second segment which consists of n′ consecutive amino acids selected in a second amino acid sequence set forth in SEQ ID NO:2 (LKHDEYNIENLQKTVWD), wherein n and n′ represent an integer number, n and n′≥3 and n+n′≥10.

2. The polypeptide of claim 1 which comprises at least 10, 11, 12, 13, 14, 15, 16, 17, 18; 19; 20; 21; 22; 23; 24; 25; 26; 27; 28; 29; 30; 31; 32; 33; 34; 35; 36; 37; 38; 39; or 40 amino acids.

3. The polypeptide of claim 1 which comprises less than 15 amino acids.

4. The polypeptide of claim 1 wherein the first segment consists of 3; 4; 5; 6; 7; 8; 9; 10; 11; 12; 13; 14; 15; 16; 17; 18; 19; 20; 21; 22; or 23 consecutive amino acids selected in the first amino acid sequence set forth in SEQ ID NO:1.

5. The polypeptide of claim 1 wherein the second segment consists of 3; 4; 5; 6; 7; 8; 9; 10; 11; 12; 13; 14; 15; 16; or 17 consecutive amino acids selected in the first amino acid sequence set forth in SEQ ID NO:2.

6. The polypeptide of claim 1 wherein the first and second segments are fused directly or via a spacer.

7. The polypeptide of claim 1 which consists of an amino acid sequence set forth in SEQ ID NO:3, 4, 5, 6, 7, 10 or 11.

8. A nucleic acid molecule encoding for the polypeptide of claim 1.

9. A vector which comprises the nucleic acid molecule of claim 8.

10. A host cell genetically transformed with the nucleic acid molecule claim 8.

11. A method of treating C3 NeF associated C3 Glomerulopathy or Barraquer-Simons syndrome in a subject in need thereof comprising administering to the subject a therapeutically effective amount of the polypeptide of claim 1 or a nucleic acid molecule encoding the polypeptide.

12. A pharmaceutical composition comprising the polypeptide of claim 1 or a nucleic acid molecule encoding the polypeptide and a pharmaceutically acceptable carrier.

13. A method for detecting the presence of C3 Nef in a sample comprising contacting the sample with the polypeptide of claim 1 under conditions that allow an immunocomplex of the polypeptide and an antibody to form, and detecting the presence of the immunocomplex.

14. The method of claim 13 wherein the sample is a blood sample.

15. The method of claim 13 wherein an assay for the detection of immunocomplexes utilizes a solid phase or substrate to which the polypeptide that is capable of inhibiting the binding of C3 NeF to C3 convertase and which comprises a first segment which consists of n consecutive amino acids selected in a first amino acid sequence set forth in SEQ ID NO:1 (GQDEENQKQCQDLGAFTESMVVF) fused to a second segment which consists of n′ consecutive amino acids selected in a second amino acid sequence set forth in SEQ ID NO:2 (LKHDEYNIENLQKTVWD), wherein n and n′ represent an integer number, n and n′≥3 and n+n′≥10, is directly or indirectly attached.

16. A kit for detecting C3Nef in a sample which comprises the polypeptide of claim 1 and means for determining binding of the polypeptide to C3Nef in a sample.


SEQUENCE LISTING (11 sequences)

SEQ ID NO: 1 - protein, 23 aa, Artificial Sequence
  Synthetic first segment
GQDEENQKQCQDLGAFTESMVVF

SEQ ID NO: 2 - protein, 17 aa, Artificial Sequence
  Synthetic second segment
LKHDEYNIENLQKTVWD

SEQ ID NO: 3 - protein, 19 aa, Artificial Sequence
  Synthetic short1
GAFTESMVVFLKHDEYNIE

SEQ ID NO: 4 - protein, 25 aa, Artificial Sequence
  Synthetic mix1
KQCQDLGAFTESMVVFKATYPKIWV

SEQ ID NO: 5 - protein, 34 aa, Artificial Sequence
  Synthetic pep1
GQDEENQKQCQDLGAFTESMVVFNIENLQKTVWD

SEQ ID NO: 6 - protein, 25 aa, Artificial Sequence
  Synthetic pep1C3bExt
GENQKQCQDLGAFTESMVVFLKHDE

SEQ ID NO: 7 - protein, 22 aa, Artificial Sequence
  Synthetic pep1BbExt
SMVVFLKHDEYNIENLQKTVWD

SEQ ID NO: 8 - protein, 19 aa, Artificial Sequence
  Synthetic pep2
GEDEEPWEQITVVYPKIWV

SEQ ID NO: 9 - protein, 22 aa, Artificial Sequence
  Synthetic pep3
GEERLDKACENNDSIGASNFTG

SEQ ID NO: 10 - protein, 13 aa, Artificial Sequence
  Synthetic Essential
SMVVFNLQKTVWD

SEQ ID NO: 11 - protein, 18 aa, Artificial Sequence
  Synthetic Essential2
GAFTESMVVFNLQKTVWD